(19) United States
(12) Patent Application Publication
(10) Pub. No.: US 2025/0102440 A1
(43) Pub. Date: Mar. 27, 2025

(54) SERS METHOD FOR ERGOTHIONEINE QUANTIFICATION BY SURFACE-AMPLIFIED RAMAN SCATTERING SPECTROSCOPY

(71) Applicant: Università degli Studi di Trieste, Trieste (IT)

(72) Inventors: Stefano FORNASARO, Trieste (IT); Alois BONIFACIO, Trieste (IT); Gioia VENTUROTTI, Trieste (IT)

(73) Assignee: Università degli Studi di Trieste, Trieste (IT)

(21) Appl. No.: 18/887,198

(22) Filed: Sep. 17, 2024

(30) Foreign Application Priority Data
Sep. 21, 2023 (IT) 102023000019458

(51) Int. Cl. G01N 21/65; C07D 235/28; G01N 33/487

(52) U.S. Cl. CPC G01N 21/658; C07D 235/28; G01N 33/487

(57) ABSTRACT

The present invention refers to a method for detecting and/or determining the amount of ergothioneine (EG) in a sample using surface-enhanced Raman scattering (SERS) spectroscopy. This method involves the use of an internal standard such as 5-amino-2-mercaptobenzimidazole. (5A2MBI).

### CROSS-REFERENCE TO RELATED APPLICATIONS

This application claims the benefit of priority from Italian Patent Application no. 102023000019458 filed Sep. 21, 2023, the disclosure of which is incorporated herein by reference.

### FIELD OF THE INVENTION

This invention pertains to the field of systems and methods for detecting the presence and/or determining the quantitative amount of a biomolecule with surface-enhanced Raman scattering (SERS) spectroscopy. In particular, relates to a SERS method suitable for the quantitative determination of ergothioneine (EG) in a sample.

### BACKGROUND OF THE INVENTION

Ergothioneine is a dietary amino acid that has attracted a lot of attention due to its remarkable antioxidant properties as well as the potential health benefits it offers [1].

Ergothioneine stands out as a highly soluble and stable compound, exhibiting exceptional resistance to heat and oxygen. Protecting cells from photochemical damage, neutralizing harmful reactive oxygen and nitrogen species (ROS, and RNS), and lowering the associated oxidative stress are all significant biological functions of this compound [2]. In addition, ergothioneine has been connected to a variety of physiological functions in humans, including neuroprotective, anti-inflammatory, and anti-aging effects. Consequently, ergothioneine has gained popularity as a potential ingredient in a variety of applications, including antioxidant formulations, skincare and cosmetics, nutraceuticals, functional foods and beverages, pharmaceuticals, biotechnology, bioengineering and clinical diagnostics [3,4]. As research uncovers its full range of benefits and applications, the uses of ergothioneine as an ingredient continue to expand. Its versatility, stability, and potent antioxidant properties make it an appealing choice for a wide range of industries seeking to develop products with improved functionality, health benefits, and consumer appeal.

Given these diverse potential applications, there is a growing demand for a specific, straightforward and simple method to measure ergothioneine in various complex matrices, including foods, cosmetics, and biological samples. Moreover, accurate quantification of ergothioneine levels in biological samples is crucial for understanding its role in oxidative stress-related diseases, such as neurodegenerative disorders, cardiovascular diseases, and cancer [1].

Existing analytical methods for ergothioneine determination include: i) capillary electrophoresis [5]; ii) High-Performance Liquid Chromatography (HPLC) [5,6]; iii) inductively coupled plasma tandem mass spectrometry (HPLC-ICP-QQQ-MS) [7]; iv) liquid chromatography-triple quadrupole tandem mass spectrometry (LC-MS/MS) [8]. However, these techniques all require complex and destructive sample preparation procedures, involve time-consuming separations, need high-purity reagents, and lack the required sensitivity for detecting ergothioneine at low concentrations in biofluids. There is a need for a reliable, rapid, sensitive, and efficient analytical method that allows for the quantitative determination of ergothioneine in various sample matrices.

One approach that is effectively used for measuring compounds at concentrations similar to those at which EG is present if biological fluids, employs surface-enhanced Raman scattering (SERS). SERS spectroscopy has emerged as a powerful analytical technique for the detection and quantification of various molecules [9]. SERS exploits the phenomenon of enhanced Raman scattering signals when analytes are adsorbed onto nanostructured metal surfaces, such as gold or silver nanoparticles. The signal enhancement arises from the excitation of localized surface plasmon resonances on the metal nanoparticles, resulting in intensified Raman signals. However, one big challenge in accurate quantification using SERS is the variability that can arise during sample preparation and analysis [10]. Factors such as variations in the SERS substrate, laser power, and experimental conditions can affect the Raman signal intensity, leading to potential inaccuracies in quantification. Including an internal standard (IS) of known concentration in the unknown sample and using its SERS spectral band intensity as a reference to the band intensity of the chemical of unknown concentration is an effective way to overcome such variation errors. However, the variability in signal enhancement across different analytes makes it difficult to identify a single compound that can serve as an appropriate IS for all cases. So far, no IS specific for ergothioneine has ever been reported in the literature.

Therefore, there is still a need for a reliable, rapid, sensitive, and efficient analytical method for the quantitative determination of EG in various matrices. More specifically, there is a need for a method that enables to measure the quantity of EG in any type of sample in the precise and accurate manner.

### SUMMARY OF THE INVENTION

To address the technical issues associated with the previously mentioned methods, the inventors with the present invention provide an effective and reliable method to rapidly detect and measure ergothioneine (EG) exploiting Surface Enhanced Raman Scattering (SERS) spectroscopy with the addition of a specific internal standard for ergothioneine. Advantageously, the method comprises the addition of a compound of formula (I), such as 5-amino-2-mercaptobenzimidazole (5A2MBI), as an internal standard for ergothioneine, which helps to mitigate the variability in the SERS signal arising from different SERS substrates.

It is an object of the invention a method for detecting and/or determining the amount of ergothioneine (EG) in a sample using surface-enhanced Raman spectroscopy (SERS), comprising the following steps:
a) adding at least one internal standard (IS) to the sample to obtain a sample that comprises at least one IS;
b) adding to the sample comprising an IS obtained in a) at least one SERS substrate;
c) exposing the sample combined with the SERS substrate obtained instep b), to a laser to generate a SERS spectrum;
   wherein the method is characterized by the fact that the internal standard has the following formula (I):

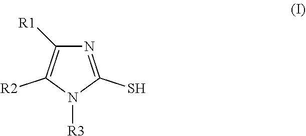

wherein:
R1 and R2 are each independently selected from C₁₋₃-alkyl, NH₂, NHCOC₁₋₃alkyl, NH(C₁₋₃-alkyl), N(C₁₋₃-alkyl)2, halogen, aryl, and heteroaryl; or
R1 and R2 are connected to form a fused aromatic or heteroaromatic ring selected from phenyl, pyrrole, imidazole, pyridine, pyrimidine, each optionally substituted with one or more substituents selected from: C₁₋₃-alkyl, NH₂, NHCOC₁₋₃alkyl, NH(C₁₋₃-alkyl), N(C₁₋₃-alkyl)2, halogen, NO₂, CN; and
R3 is H, CH₃, or NH₂.

In an embodiment said internal standard has the formula (I) wherein R1 and R2 are linked forming a fused aromatic ring, in particular a phenyl, optionally substituted with one or more substituents, preferably substituted with NH₂ and/or wherein R3 is H.

In a preferred embodiment, the internal standard has the following formula (II):

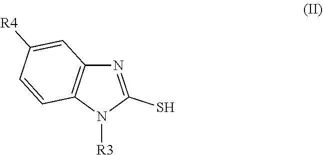

wherein R3 is H, CH₃, or NH₂; and
R4 is NH₂, NO₂, H, or halogen.

Preferably, R3 is H and preferably, R4 is NH₂.

In an even more preferred embodiment, the internal standard is 5-amino-2-mercaptobenzimidazole(5A2MBI). According to one aspect of the invention in the step a), the internal standard is added at a concentration comprised between 2.5 nM and 25 μM, preferably at a concentration of 0.25 μM.

According to one aspect of the invention in step c), the laser is used at a wavelength comprised between 600 and 850 nm, preferably 785 nm.

According to one aspect of the invention, the method comprises an additional step d) wherein the presence and/or quantity of EG in the sample is determined by comparing the characteristic spectral band of EG at 484 cm⁻¹, with the characteristic spectral band of the internal standard. For 5A2MBI, the characteristic spectral band is at 391-393 cm⁻¹.

According to one aspect of the invention, the SERS substrate comprises at least one nanostructure selected from spherical nanoparticles, nanorods, nanostar, and nanoplate.

According to one aspect of the invention, the SERS substrate comprises at least one nanostructure of silver or gold reduced with citrate or another reducing agent, or at least one nanostructure of silver or gold obtained by laser ablation.

In a preferred embodiment, said SERS substrate comprises citrate-reduced silver nanoparticles (c-AgNPs).

According to one aspect of the invention, the sample is selected from an isolated biological sample, such as a biological fluid, a food product, and a cosmetic.

In a preferred embodiment, said sample is a biological fluid, preferably serum.

According to one aspect of the invention, the method also comprises a step of deproteinization of the sample prior to step a).

According to one aspect of the invention, after step b), the sample combined with the SERS substrate is deposited, preferably in the form of one or more drops, onto a support and is left to dry until desiccation.

According to an aspect of the invention, said support is chosen from a calcium fluoride plate and a glass plate, preferably coated with at least one layer of aluminum or with at least one layer of aluminum and at least one layer of parafilm.

It is another object of the invention the use of a compound of formula (I) or (II) as described above, preferably of 5A2MBI, as an internal standard in a SERS method for measuring the concentration of EG in a sample.

### BRIEF DESCRIPTION OF THE DRAWINGS

FIG. 1. General scheme.

FIG. 2. Dependence of the SERS signal on the concentration of EG in human serum. Five replicates were considered for each concentration level (0, 1, 2, 3, 4, 5, 6, 7, 8, 9, 10 μM) over two different days. Concentration of the IS: 0.25 μM.

FIG. 3. Calibration curve for the quantification of EG in human serum. The confidence and prediction bands are shown as shaded areas, dark gray and light gray, respectively. The limit of detection (LOD) and the limit of quantification (LOQ) are reported at the top left. The value of the coefficient of determination (R²) is 0.98.

FIG. 4. General scheme.

FIG. 5. Dependence of the SERS signal on the concentration of EG in human serum. Five replicates were considered for each concentration level (0, 0.2, 0.4, 0.6, 0.8, 1, 1.2, 1.4, 1.6, 1.8, 2 μM) over two different days. Concentration of the IS: 0.25 μM.

FIG. 6. Calibration curve for the quantification of EG in human serum. The confidence and prediction bands are shown as shaded areas, dark gray and light gray, respectively. The limit of detection (LOD) and the limit of quantification (LOQ) are reported at the top left. The value of the coefficient of determination (R²) is 0.97.

### DETAILED DESCRIPTION OF THE INVENTION

### Definitions

In the context of the present invention, “ergothioneine” refers to an amino acid known by the IUPAC name (2S)-3-(2-Sulfanylidene-2,3-dihydro-1H-imidazol-4-yl)-2-(trimethylazaniumyl)-propanoate. It is also indicated herein with the acronym “EG” or “EGT”.

In the context of the present invention, “Surface Enhanced Raman Scattering” (SERS) refers to an analytical method for the detection and quantification of a compound of interest in a sample that exploits the phenomenon of the enhancement of Raman scattering when the analytes are adsorbed onto nanostructured metallic surfaces. “SERS” is used herein as a synonym.

In the context of the present invention, “internal standard” refers to a chemical compound that is added in a known quantity to a sample, to a blank, and to a calibration standard in a chemical analysis.

In the context of the present invention, “SERS substrate” refers to a substrate suitable for use in the SERS methodology, comprising a nanostructured surface of at least one noble metal. Said metal can be chosen from gold, silver, copper, or the combinations thereof. In particular, said SERS substrate may comprise multiple nanostructures, where a nanostructure is defined as a particle with a diameter or at least one length typically comprised from 1 to 100 nm, for example, 50 nm. Said nanostructure can have different shapes, particularly a spherical shape, a rod shape (nanorod), or a star shape (nanostar). Said nanostructures can be in aqueous dispersion or deposited on a support that may comprise paper, silicon, or other suitable materials. In a preferred embodiment, said nanostructure is a nanoparticle.

In the context of the present invention, “limit of detection (LOD)” refers to the lowest concentration of EG in a sample that can be consistently detected with a stated level of confidence (typically 95%).

In the context of the present invention, “limit of quantification (LOQ)” refers to the lowest concentration of EG in a sample that can be quantitatively detected with acceptable accuracy and precision (10% error).

In the context of the present invention, “laser” refers to an optoelectronic device capable of emitting a coherent beam of light.

In the context of the present invention, “isolated biological sample” refers to a sample that has been previously isolated from a subject. In particular, said sample can be, for example, a biological fluid or a tissue.

In the context of the present invention, “biological fluid” or “biofluid” refers to any biological fluid, such as blood, plasma, serum, saliva, or urine.

The method of the invention has been validated on samples containing different concentrations of EG, achieving narrow confidence and prediction bands, indicating that the model has a low error.

In a preferred embodiment, the SERS substrate is a nanostructure selected from spherical nanoparticle, nanorod, nanostar, or nanoplate. Said nanostructure can be, for example, made of silver or gold reduced with citrate or another reducing agent, or a silver or gold nanostructure obtained by laser ablation or another suitable method. Preferably, said SERS substrate comprises citrate-reduced silver nanoparticles. (c-AgNPs). Said nanoparticles can be, for example, in aqueous dispersion or deposited on paper.

Said citrate-reduced silver nanoparticles (c-AgNPs) can be synthesized using the Lee-Meisel method (see ref. [11]), wherein AgNO₃ is dissolved in deionized water (DI), brought to a boil under magnetic stirring, and then drops of trisodium citrate solution are added. In a preferred form of realization, the SERS substrate, preferably c-AgNPs, is mixed with the sample of interest in a ratio comprised between 1:1 and 1:9, preferably at 1:1 ratio.

In a further preferred embodiment, the method comprises a phase of sample preparation prior to step a). Said phase of sample preparation may comprise a deproteinization step, i.e. the partial or total reduction of the protein content present in the sample. Deproteinization can be carried out using any known method, for example, using centrifugal filters.

For example, a sample solution is added to the filters and centrifuged to obtain a filtered sample. Deproteinization enhances the effectiveness of SERS when using spherical metallic nanoparticles as SERS substrates, as proteins hinder the aggregation of nanoparticles, which is necessary to form nanoparticle clusters with the plasmonic properties required to achieve a strong SERS effect.

The internal standard has the formula (I) as described above. In such a formula (I), preferably, R1 and R2 are linked to form a fused aromatic ring, particularly a phenyl, optionally substituted with one or more substituents, preferably substituted with NH₂. The substituents can be in any suitable position on the ring, preferably one substituent is in position 5.

R3 is preferably H.

In a preferred embodiment, the internal standard is 5-amino-2-mercaptobenzimidazole (5A2MBI).

Compounds of formula (I) or (II) are commercially available or can be obtained using common synthesis methods according to general knowledge in the field.

The internal standard is added to the sample in step a) at a concentration that varies based on the characteristics of the SERS substrate and the analyzed matrix.

The expert in the field is able to determine the appropriate concentration based on general knowledge in the field. In an embodiment, the concentration of the internal standard is comprised between 2.5 nM and 25 μM, preferably is at 0.25 μM.

In a further preferred embodiment, after step b), the sample combined with the SERS substrate is deposited, preferably in the form of one or more drops, onto a support and is is left to dry until completely dry (desiccated). Preferably, the sample is deposited on the support in the form of a droplet, and laser measurements are taken in the peripheral ring (coffee ring) of the dried droplet. The term “coffee ring” refers to a region of the dried droplet wherein the deposited nanoparticles have a higher density compared to the rest of the dried droplet (“coffee ring effect”).

Preferably, said droplet has a volume greater than 1 μL, for example comprised between 30 and 70 μL, preferably 50 μL. Said support can be, for example, made of glass, quartz, calcium fluoride, steel, or aluminum. Preferably, said support is selected from a calcium fluoride plate and a glass plate, preferably coated with a layer of aluminum or with a layer of aluminum and a layer of parafilm. This last embodiment is particularly advantageous as it avoids spectral interferences from Raman bands or substrate fluorescence; moreover, the hydrophobic characteristic of the parafilm prevents the diffusion of the liquid sample, ensuring the formation of a thick hemispherical substrate droplet.

In passage c), the laser can be used at any wavelength. The wavelength can be chosen by the expert in the field depending on the matrix to be analyzed. Preferably, the laser is used with a wavelength comprised in the range between 600 and 850 nm, and more preferably at a wavelength of about 785 nm. Any laser equipment known in the field can be used.

In an exemplary embodiment of the present invention, in step c), the laser is used with a power of 1% (10 mV), an exposure time of 10 seconds, and/or a number of accumulations equal to one. Said power advantageously allows for the avoidance of damage to the sample.

In a preferred embodiment of the invention, the amount of EG in the sample is determined by analyzing the SERS spectrum obtained in step c) of the method, comparing the characteristic spectral band of EG at 484 cm⁻¹ with the characteristic spectral band of 5A2MBI at 391-393 cm⁻¹. In other embodiments, the characteristic spectral band of the EG is compared with the characteristic spectral band of other compounds of formula (I) or (II), according to what is known in the field. From the comparison of the bands, the quantity of EG can be determined according to the methods known in the field.

Preferably, the method is used in a sample that contains an EG concentration of less than 20 μM, preferably less than 10 μM.

Unless otherwise stated here, the SERS method can be performed as is known in the field. For example, see references [9, 12].

It is also an object of the invention the use of a compound of formula (I) or (II), preferably 5A2MBI, as an internal standard in a SERS method for measuring the concentration of EG in a sample. The expert in the field is able to obtain a calibration curve that allows for the determination of EG concentration in a sample using a compound of formula (I) or (II), preferably 5A2MBI, as an internal standard in a SERS method.

The method of invention can be advantageously applied in the cosmetic, food, nutraceutical, and pharmaceutical industries, as well as in biotechnology, bioengineering, and clinical diagnostics.

In an exemplary embodiment of the present invention, the method for determining the amount of ergothioneine (EG) in a sample using surface-enhanced Raman scattering (SERS) spectroscopy comprises adding an internal standard to the sample, such as 5-amino-2-mercaptobenzimidazole (5A2MBI) (step a). Subsequently, to said sample comprising IS obtained in (a) is added a SERS substrate (step b). Said sample obtained in (b) is then exposed to a laser with a wavelength of 785 nm to generate a SERS spectrum (step c).

In a further exemplary embodiment of the present invention, IS is 5A2MBI and is added to the sample at a concentration of 0.25 μM and is then mixed with the c-AgNPs substrate. The concentration of IS 5A2MBI is typically determined based on the intensity value of the internal standard band, which must be less intense than that of ergothioneine.

In a further exemplary embodiment of the present invention, the SERS spectrum of the sample in (c) is acquired using a Raman spectrometer, for example, the portable i-Raman plus spectrometer, equipped with a 785 nm laser (output 400 mV) and connected to a microscope, such as a compact microscope with Olympus 20× optics.

In a further exemplary embodiment of the present invention, the recording of the SERS spectrum is carried out using software, for example, BWSpec™ version 4.03_23_c in the range of 62-3202 cm⁻¹. Said software BWSpec™ allows for the acquisition of a background (dark) signal before data collection and subtracts it from the collected data. Furthermore, the SERS spectrum recording of the sample may comprise a step of the wavelength calibration, which is verified before and during each recording session by recording the spectrum of at least one standard reference, such as a spectrum of paracetamol and silicon.

In a further exemplary embodiment of the present invention, the method for determining the presence and quantifying ergothioneine in the sample comprises an analysis of the SERS spectrum obtained in (c), comparing the spectral band at 484 cm⁻¹, characteristic of ergothioneine, with the spectral band at 391-393 cm⁻¹, characteristic of 5A2MBI.

The invention will now be illustrated with the help of examples.

### EXAMPLES

### Materials and Methods

The method is broken down into a series of very straightforward steps, which are described as follows:
1. obtaining a sample of biofluid;
2. adding the IS, 5A2MBI to the sample;
3. combining the sample with a SERS substrate;
4. exposing the combined sample and the SERS substrate to laser illumination at 785 nm to generate a SERS spectrum;
5. analyzing the SERS spectrum to determine the presence of ergothioneine in the serum sample by comparing the spectral band at 484 cm⁻¹ that is characteristic of ergothioneine with the 5A2MBI characteristic band at 391-393 cm⁻¹.

In step 2, 0.25 μM of 5A2MBI is used with c-AgNPs substrate. The decision of the concentration shall be made based on the intensity of the IS band, that must be less intense with respect to the one of EG. Normalization with a high intensity value for the IS band may underestimate the real measures.

Ultrapure deionized (DI) water of 18.2 MΩ cm resistivity at 25° C. was used throughout the experiments and it was obtained using a Millipore Milli-Q system (Merck, Germany). Buffer solution was prepared by dissolving a tablet of PBS in 200 mL of DI water, under magnetic stirring (for 20 min). L-(+)-Ergothioneine (EG) stock solution (10 mM) was prepared by solubilizing 2.3 mg of EG in 1 mL of buffer solution. Adenine solution was prepared dissolving the powder in NaOH 1 M to obtain a solution of 5 mM, successively diluted in buffer solution to arrive at a final concentration of 10 μM. Stock solution of 5-amino-2-mercaptobenzimidazole (5-A-2MBI) 10 mM was prepared dissolving 1.65 mg of powder in 1 mL of methanol. After this step it was possible to mix it with buffer solution to prepare the intermediate dilutions before adding it to the samples.

Citrate-reduced silver nanoparticles (c-AgNPs) were synthesized using the Lee-Meisel method [11]. Briefly, 45 mg of AgNO₃ were dissolved in 250 mL of DI water and heated to boiling and under magnetic stirring. Successively 5 mL of a 1% sodium citrate tribasic solution were added dropwise to the boiling solution. The solution was kept boiling and stirring for 1 h in conditions of complete darkness. The result is a greenish grey solution. The c-AgNP were stored in dark at room temperature and were stable for months. All colloids have been characterized by UV-Visible absorption spectroscopy after each preparation, using UV-visible spectroscopy (Cary60, Agilent Technology). The extinction band maxima were between 405 and 410 nm, corresponding to an average particle size of 50 nm.

To assess the activity and to evaluate the batch-to-batch variability of c-AgNPs, a set of SERS measurements have been performed using adenine as reference analyte [12]. This metabolite gives an intense and well-defined spectrum because of its high affinity for the nanoparticles surface, and, in addition, it is not light or temperature sensitive, it has low toxicity, and it is inexpensive.

### SERS Instrumentation

SERS spectra were collected at room temperature (22±0.5° C.) with a portable i-Raman plus spectrometer (B&W Tek, Plainsboro, NJ, USA) equipped with a 785 nm laser (output 400 mV) and connected with a compact microscope mounting an Olympus optics 20× with a spot size of 108 μm, NA of 0.40. The spectral acquisition was performed using the BWSpec™ version 4.03_23_c (B&W Tek., Newark, DE, USA) software in the Raman shift range of 62-3202 cm⁻¹. The BWSpec™ software allowed for the collection of a background signal (dark) before data acquisition and its subtraction from the collected data. Wavenumber calibration was checked before and during every spectral acquisition session by collecting a spectrum of paracetamol and silicon as standard references.

### Results

### Example 1

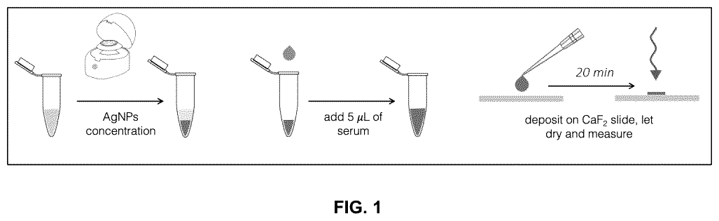

Sample preparation is depicted in FIG. 1. c-AgNPs were concentrated 10 times in volume with benchtop centrifuge (15 min×11337×g), starting from a volume of 1 mL. The supernatant (900 μl) was removed, and the remaining pellet of nanoparticles was suspended again in 100 μl. At this point 5 μL of concentrated nanoparticles were distributed in different PCR tubes of 500 μl, one for each sample, and mixed with 5 μL of sample (ratio 1:1). A drop of 5 μL was deposited on a slide made of CaF2 or glass covered in aluminum foil. It has been demonstrated during this phase of experiments, that the nature of the substrate doesn't have influence on the SERS signal. Glass micro slides covered in aluminum foil are less expensive and easier to use if compared to CaF2 slides and for this reason they were chosen for the successive experiments. The drops on the slide remain to dry for 20 minutes at room temperature; after this time measurements were obtained in the peripheral ring of the dried drop, a distinctive zone named “coffee ring”, where the signal of the analytes is more intense. Five measurements are taken for each drop to reduce the effect of variability intra-substrate: laser power was 1% (10 mV), to avoid damages to the samples, 10 seconds of time of exposition, number of accumulation equal to one.

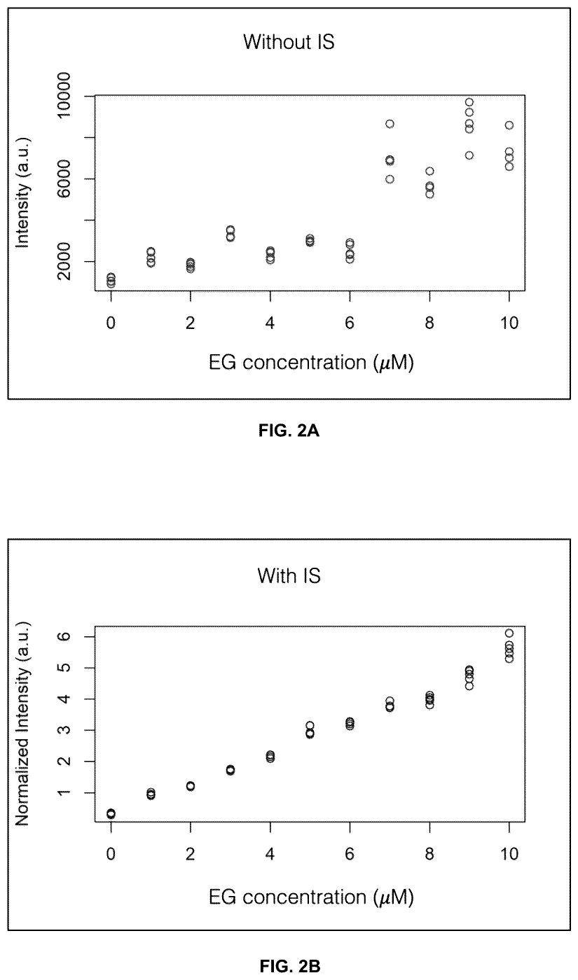

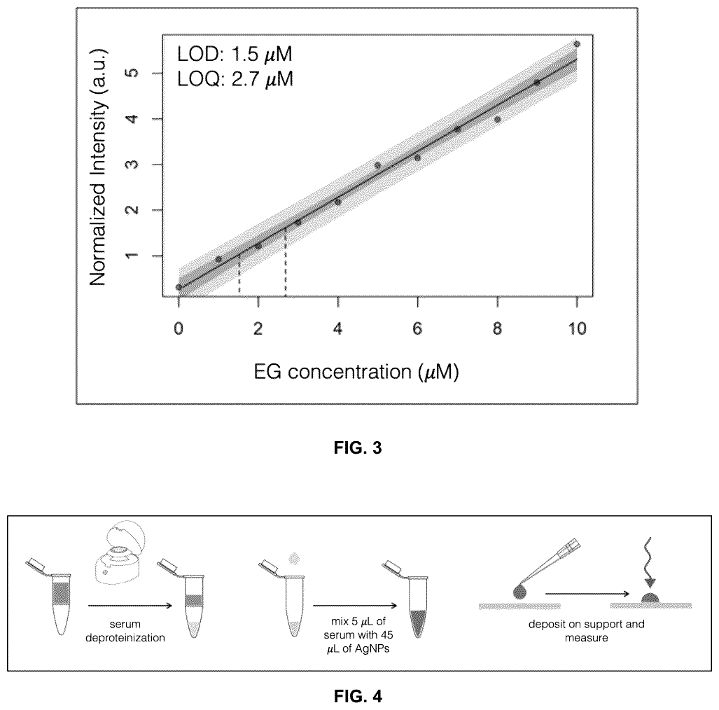

The results of two experiments using c-AgNPs mixed with the sample of interest and then dried on a support are reported in FIG. 2. The change apported by normalization through internal standard is evident, with values much less variable for each point of concentration, and an apparent linear relation between normalized intensity and EG concentration. FIG. 3 reported the complete calibration in the range between 1 to 10 μM with the respective figures of merit necessary for the validation of the calibration model, to assess the prediction ability and define the quality of the analytical method. The adjusted R-squared of 0.98, and the narrow bands of error and prediction, indicate that the model is good. Limit of detection (LOD), defined as the lowest concentration of an analyte in a sample that can be consistently detected with a stated confidence level (typically 95%) was 1.4 μM and limit of quantification (LOQ), defined as the lowest analyte concentration that can be quantitatively detected with an acceptable accuracy and precision (10% error) was 2.7 μM.

### Example 2

Sample preparation is depicted in FIG. 4. 25 μL of human serum (Human Serum; Sigma Aldrich; product no. H4522) enriched with different EG concentrations were mixed with 25 μL of a colloidal dispersion of c-Ag (c-AgNPs) in a PCR tube of 0.5 mL. Immediately after mixing, a change in the color can be observed, indicating a partial nanoparticles aggregation due to the absorption of the analytes on the metal surface. The resulting 50 μL droplet was rapidly deposited under the microscope objective on glass micro slides (25×75 mm), previously covered in aluminum foil and parafilm, that was fitted onto the portable microscope stage. This setting was chosen to avoid spectral interference from Raman bands or fluorescence from the substrate. Moreover, the hydrophobic feature of parafilm avoided the spread of the liquid sample, ensuring a thick semi-spherical drop. The choice to use a droplet volume of 50 μL had purely practical reason: it is easier to handle, but in case of necessity it is possible to reduce the volume up to 30 μL.

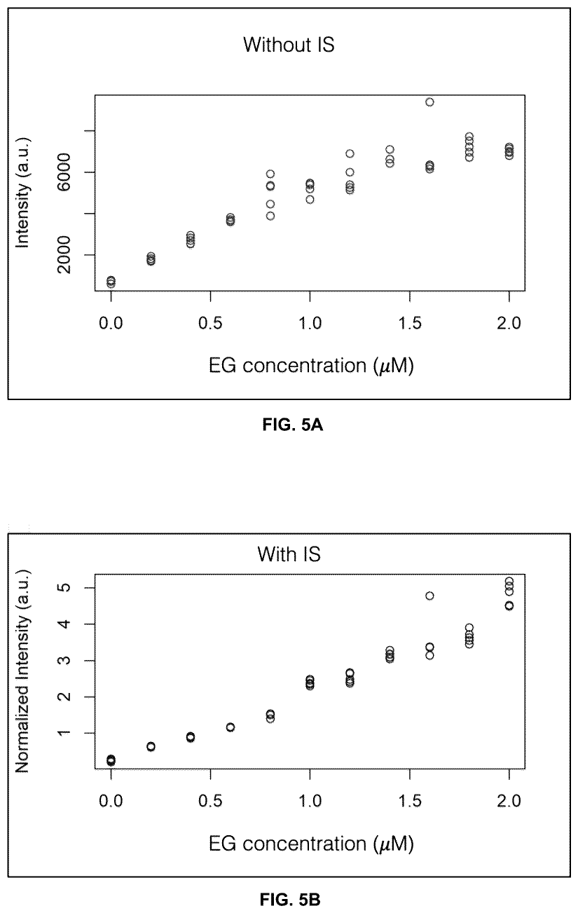

The sample preparation included a step of deproteinization using centrifugal filters (3 kDa Vivaspin® 0.5 mL, Sartorius, UK). The filters were cleaned before use through centrifugation with DI water (11337×g×15 min., 2 times); sample solution was added after this procedure and centrifuged (2 cycle of 20 min.×8117×g) to obtain filtered sample at the base of the filter's Eppendorf. The filtered sample was used for measurements and was kept at low temperature (4° C.) to avoid degradation. The effect of filtration on the spectra suggests that the proteins present in serum (collectively referred to as serum proteins) prevent surface enhancement from tacking place, and thus the observation of SERS spectra [13]; the effect is probably due to the absence of nanoparticles aggregation in presence of serum proteins. When using spherical metal nanoparticles as SERS substrates, at least a partial aggregation is necessary to form nanoparticles clusters having the suitable plasmonic properties in order to obtain a strong SERS effect. Aqueous metal colloids are stabilized by electrostatic repulsion, which keeps nanoparticles apart, thus preventing their aggregation upon collision with one another. The stabilizing surface charge mostly originates from charged chemical species directly absorbed onto the nanoparticle surface (like citrate ions for c-AuNP and c-AgNP or chloride ions for h-AgNP). But, if it is introduced a high electrolyte content, such as serum or plasma, stabilizing surface charges are “shielded” by the ions in solution, causing a rapid aggregation of nanoparticles. However, serum proteins (mostly albumin) are known to immediately adsorb onto the metal nanoparticles surface to form a layer called “protein corona”, establishing a steric repulsion between nanoparticles which prevents them from colliding with one another, thus avoiding aggregation. Results are reported in FIG. 5. The change apported by normalization through internal standard is evident, with values much less variable for each point of concentration, and an apparent linear relation between normalized intensity and EG concentration.

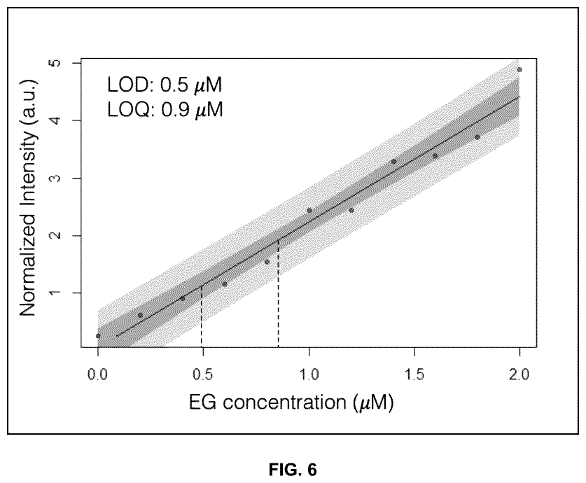

FIG. 6 reported the complete calibration in the range between 0.2 to 2 μM with the respective parameters necessary for the validation of the calibration model, to assess the prediction ability and define the quality of the analytical method. A value of 0.97 for the coefficient of determination R² and narrow confidence and prediction bands indicate that the model has a low error. The limit of detection (LOD), defined as the lowest concentration of an analyte in a sample that can be detected consistently with a stated confidence level (typically 95%) was 0.5 μM, while the limit of quantification (LOQ), defined as the lowest concentration of an analyte that can be quantitatively detected with acceptable accuracy and precision (10% error) was 0.9 μM.

### REFERENCES

ADDIN ZOTERO_BIBL {“uncited”:[ ],“omitted”:[ ],“custom”:[ ]}CSL_BIBLIOGRAPHY [1]I. Borodina, L. C. Kenny, C. M. McCarthy, K. Paramasivan, E. Pretorius, T. J. Roberts, S. A. van der Hoek, D. B. Kell, The biology of ergothioneine, an antioxidant nutraceutical, Nutr. Res. Rev. (2020) 1-28. https://doi.org/10.1017/SO954422419000301.
[2]I. K. Cheah, B. Halliwell, Ergothioneine; antioxidant potential, physiological function and role in disease, Biochimica et Biophysica Acta (BBA)—Molecular Basis of Disease. 1822 (2012) 784-793. https://doi.org/10.1016/j.bbadis.2011.09.017.
[3]I. K. Cheah, B. Halliwell, Ergothioneine, recent developments, Redox Biology. 42 (2021) 101868. https://doi.org/10.1016/j.redox.2021.101868.
[4]B. Halliwell, I. K. Cheah, R. M. Y. Tang, Ergothioneine—a diet-derived antioxidant with therapeutic potential, FEBS Lett. 592 (2018) 3357-3366. https://doi.org/10.1002/1873-3468.13123.
[5]S. Sotgia, E. Pisanu, G. Pintus, G. L. Erre, G. A. Pinna, L. Deiana, C. Carru, A. Zinellu, Plasma L-ergothioneine measurement by high-performance liquid chromatography and capillary electrophoresis after a pre-column derivatization with 5-iodoacetamidofluorescein (5-IAF) and fluorescence detection, PLoS One. 8 (2013) e70374. https://doi.org/10.1371/journal.pone.0070374.
[6]M. Muda, F. Pelizzoni, G. Sello, E. Mussini, Determination of ergothioneine in red blood cells by high-performance liquid chromatography, J Chromatogr. 434 (1988) 191-195. https://doi.org/10.1016/0378-4347(88)80074-4.
[7]N. Kroepfl, K. A. Francesconi, T. Schwerdtle, D. Kuehnelt, Selenoneine and ergothioneine in human blood cells determined simultaneously by HPLC/ICP-QQQ-MS, J. Anal. At. Spectrom. 34 (2019) 127-134. https://doi.org/10.1039/C₈JA00276B.
[8]L.-Z. Wang, W.-L. Thuya, D. S.-L. Toh, M. G.-L. Lie, J.-Y. A. Lau, L.-R. Kong, S.-C. Wan, K.-N. Chua, E. J.-D. Lee, B.-C. Goh, Quantification of L-ergothioneine in human plasma and erythrocytes by liquid chromatography-tandem mass spectrometry, J Mass Spectrom. 48 (2013) 406-412. https://doi.org/10.1002/jms.3150.
[9]J. Langer, D. Jimenez de Aberasturi, J. Aizpurua, R. A. Alvarez-Puebla, B. Auguié, J. J. Baumberg, G. C. Bazan, S. E. J. Bell, A. Boisen, A. G. Brolo, J. Choo, D. Cialla-May, V. Deckert, L. Fabris, K. Faulds, F. J. Garcia de Abajo, R. Goodacre, D. Graham, A. J. Haes, C. L. Haynes, C. Huck, T. Itoh, M. KAll, J. Kneipp, N. A. Kotov, H. Kuang, E. C. Le Ru, H. K. Lee, J.-F. Li, X. Y. Ling, S. A. Maier, T. Mayerh6fer, M. Moskovits, K. Murakoshi, J.-M. Nam, S. Nie, Y. Ozaki, I. Pastoriza-Santos, J. Perez-Juste, J. Popp, A. Pucci, S. Reich, B. Ren, G. C. Schatz, T. Shegai, S. Schlücker, L.-L. Tay, K. G. Thomas, Z.-Q. Tian, R. P. Van Duyne, T. Vo-Dinh, Y. Wang, K. A. Willets, C. Xu, H. Xu, Y. Xu, Y. S. Yamamoto, B. Zhao, L. M. Liz-Marzán, Present and Future of Surface-Enhanced Raman Scattering, ACS Nano. 14 (2020) 28-117. https://doi.org/10.1021/acsnano.9b04224.
[10]R. Goodacre, D. Graham, K. Faulds, Recent developments in quantitative SERS: Moving towards absolute quantification, TrAC Trends in Analytical Chemistry. 102 (2018) 359-368. https://doi.org/10.1016/j.trac.2018.03.005.
[11]P. C. Lee, D. Meisel, Adsorption and surface-enhanced Raman of dyes on silver and gold sols, J. Phys. Chem. 86 (1982) 3391-3395. https://doi.org/10.1021/j100214a025.
[12]S. Fornasaro, F. Alsamad, M. Baia, L. A. E. Batista de Carvalho, C. Beleites, H. J. Byrne, A. Chiad6, M. Chis, M. Chisanga, A. Daniel, J. Dybas, G. Eppe, G. Falgayrac, K. Faulds, H. Gebavi, F. Giorgis, R. Goodacre, D. Graham, P. La Manna, S. Laing, L. Litti, F. M. Lyng, K. Malek, C. Malherbe, M. P. M. Marques, M. Meneghetti, E. Mitri, V. Mohaček-Grošev, C. Morasso, H. Muhamadali, P. Musto, C. Novara, M. Pannico, G. Penel, O. Piot, T. Rindzevicius, E. A. Rusu, M. S. Schmidt, V. Sergo, G. D. Sockalingum, V. Untereiner, R. Vanna, E. Wiercigroch, A. Bonifacio, Surface Enhanced Raman Spectroscopy for Quantitative Analysis: Results of a Large-Scale European Multi-Instrument Interlaboratory Study, Anal. Chem. 92 (2020) 4053-4064. https://doi.org/10.1021/acs.analchem.9b05658.
[13]A. Bonifacio, S. D. Marta, R. Spizzo, S. Cervo, A. Steffan, A. Colombatti, V. Sergo, Surface-enhanced Raman spectroscopy of blood plasma and serum using Ag and Au nanoparticles: a systematic study, Analytical and Bioanalytical Chemistry. 406 (2014) 2355-2365. https://doi.org/10.1007/s00216-014-7622-1.

1. A method for detecting and/or determining the amount of ergothioneine (EG) in a sample using surface-enhanced Raman spectroscopy (SERS), comprising:
a) adding an internal standard (IS) to a sample to obtain a sample including an IS;
b) adding a SERS substrate to the sample including the IS obtained in a);
c) exposing the sample combined with the SERS substrate obtained in step b), to a laser to generate a SERS spectrum;
wherein the internal standard has the formula (I): 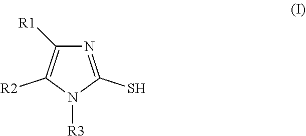
wherein:
R1 and R2 are each independently selected from C₁₋₃-alkyl, NH₂, NHCOC₁₋₃ alkyl, NH(C₁₋₃-alkyl), N(C₁₋₃-alkyl)2, halogen, aryl and heteroaryl;
or R1 and R2 are linked forming a fused aromatic or heteroaromatic ring selected from phenyl, pyrrole, imidazole, pyridine, pyrimidine, each optionally substituted with one or more substituents selected from: C₁₋₃-alkyl, NH₂, NHCOC₁₋₃alkyl, NH(C₁₋₃-alkyl), N(C₁₋₃-alkyl)2, halogen, NO₂, CN; and
R3 is H, CH₃, or NH₂.

2. The method according to claim 1, wherein R1 and R2 are linked forming a fused aromatic ring, optionally substituted with one or more substituents, and/or wherein R3 is H.

3. The method according to claim 2, wherein the fused aromatic ring is a phenyl.

4. The method according to claim 2, wherein the fused aromatic ring is substituted with NH₂.

5. The method according to claim 1, wherein said internal standard has formula (II): 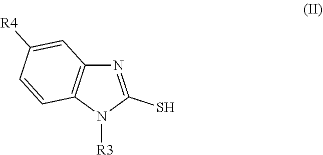
wherein R3 is H, CH₃, or NH₂; and
R4 is NH₂, NO₂, H, or halogen.

6. The method according to claim 5, wherein R3 is H and R4 is NH₂.

7. The method according to claim 1, wherein the internal standard is 5-amino-2-mercaptobenzimidazole (5A2MBI).

8. The method according to claim 1, wherein in step a) the concentration of the internal standard is between 2.5 nM and 25 μM.

9. The method according to claim 8, wherein the concentration is 0.25 μM.

10. The method according to claim 1, wherein in step c) the laser is used at a wavelength of between 600 and 850 nm.

11. The method according to claim 1, further comprising a step d) wherein the presence and/or the amount of EG in the sample is determined by comparing the characteristic spectral band of EG at 484 cm⁻¹, with the characteristic spectral band of the internal standard.

12. The method according to claim 11, wherein the internal standard is 5A2MBI and its characteristic spectral band is at 391-393 cm⁻¹.

13. The method according to claim 1, wherein the SERS substrate comprises a nanostructure selected from a spheroidal nanoparticle, nanorod, nanostar, and nanoplate.

14. The method according to claim 1, wherein the SERS substrate comprises a nanostructure of silver or gold reduced with citrate or other reducing agent, or a nanostructure of silver or gold obtained by laser ablation.

15. The method according to claim 1, wherein said SERS substrate comprises citrate-reduced silver nanoparticles (c-AgNPs).

16. The method according to claim 1, wherein the sample is chosen from an isolated biological sample, a food or a cosmetic.

17. The method according to claim 1, wherein said sample is a biological fluid.

18. The method according to claim 1, further comprising a step of deproteinization of the sample prior to step a).

19. The method according to claim 1, wherein after step b), the sample combined with the SERS substrate is deposited on a support and is left to dry until desiccation.

20. The method according to claim 19, wherein said support is chosen between a calcium fluoride plate or a glass plate optionally coated with a layer of aluminum or a layer of aluminum and a layer of parafilm.
